(19) United States
(12) Patent Application Publication
(10) Pub. No.: US 2007/0239277 A1
(43) Pub. Date: Oct. 11, 2007

(54) Intervertebral implant

(75) Inventors: Jens Beger, Tuttlingen (DE); Juergen Wegmann, Stockach (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/732,697

(22) Filed: Apr. 3, 2007

(30) Foreign Application Priority Data
Apr. 6, 2006 (DE) 10 2006 016 986.7

(51) Int. Cl. A61F 2/44

(52) U.S. Cl. 623/17.13

(57) ABSTRACT

In an intervertebral implant with at least one contact element having a vertebral body contact face and with a swellable core, which is connected to the contact element on the side remote from the vertebral body contact face, wherein the contact element comprises a support, on one side of which the vertebral body contact face is arranged and which on its other side carries projections surrounded by the material of the core, in order to improve the connection between the core, on one side, and the support, on the other, it is proposed that the projections have elongated, thread-like or rod-like anchoring sections, which extend at a spacing from the support and are freely accessible from all sides.

The present disclosure relates to the subject matter disclosed in German Application No. 2006 016 986.7 of Apr. 6, 2006, which is incorporated herein by reference in its entirety and for all purposes.

The invention relates to an intervertebral implant with at least one contact element having a vertebral body contact face and with a swellable core, which is connected to the contact element on the side remote from the vertebral body contact face, wherein the contact element comprises a support, on one side of which the vertebral body contact face is arranged and which on its other side carries projections surrounded by the material of the core.

Intervertebral implants that have a hydrogel core arranged between two end plates made of metal or plastic are used for intervertebral disc reconstruction. This hydrogel core is composed of a swellable material that can increase in volume by absorbing water and therefore has a certain structural similarity to a natural intervertebral disc. The permanent connection between such a core made of a relatively soft material, on one side, and the end plates made of a rigid material, on the other, is difficult to achieve. For example, it is known to allow the swellable material to migrate into a porous structure of the end plates (U.S. Pat. No. 5,314,478). However, this is only possible when the end plates have a corresponding porous structure. Moreover, problems can arise therein as a result of the porous structure being filled by the swellable material to such an extent that the growth of bone substance into the porous structure is impeded.

Intervertebral structures are also known, in which core materials are connected to the end plates by means of projections, these projections being configured as ribs or ridges (U.S. Pat. No. 5,674,294) or as screw-in plates (EP 0317972 A1). In this case, connection always occurs only in a part-section of the contact surface between the core material and the material of the end plates or contact elements.

It is an object of the invention to improve a generic intervertebral implant so as to improve the contact between a softer core material, on one side, and a contact element of the intervertebral implant resting against this, on the other.

This object is achieved according to the invention with an intervertebral implant of the above-described type in that the projections have anchoring sections, which are elongated, thread-like or rod-like, and which extend at a spacing from the support and are freely accessible from all sides. The soft material surrounds these thread- or rod-like sections on all sides because of their free accessibility from all sides, therefore the anchoring sections are completely embedded in the softer material and thus form an intermediate member between the support and the core material that connects these materials permanently and very firmly to one another.

The anchoring sections preferably extend substantially parallel to the vertebral body contact face and at a spacing from the side of the support remote from the vertebral body contact face, so that a closed layer of the softer core material can be arranged between the anchoring sections and the support.

It is particularly advantageous if substantially the entire surface of the support has anchoring sections. Then, a connection between the support, on one side, and the core material, on the other, can be created by means of the entire contact surface of the two materials, i.e. the anchoring arrangement is not restricted to only certain regions of the contact surface.

A plurality of anchoring sections can extend parallel to one another.

In a particularly preferred embodiment it is provided that various anchoring sections intersect.

In particular, intersecting anchoring sections can form a net-like structure, i.e. a structure in the manner of a woven or knitted fabric.

It is particularly advantageous if the support has frame-like side parts, on which the anchoring sections are held. The anchoring sections are then practically clamped between these side parts, and this applies in particular when using flat, net-like structures.

For example, the anchoring parts can penetrate into holes of the frame-like side parts and can be secured in these. The anchoring parts can be screwed into the holes to secure them.

In another embodiment it is provided that the anchoring parts are secured in the holes by means of enlargements or knots on the outside of the side parts.

In a further embodiment the anchoring parts are made of swellable material and surround spigots and sockets in the holes upon swelling. Therefore, a positive-locking fixture of the anchoring parts in the holes results when water is absorbed.

In a further preferred embodiment, the anchoring sections are U-shaped with two legs, which extend transversely to the supports and secure the anchoring sections on the support, and with a web connecting these legs. In the case of these anchoring sections, the web normally extends substantially in a plane arranged parallel to the vertebral body contact face and at a spacing from the support, in particular the legs and the web can extend substantially rectilinearly.

In this case, it is favourable if the legs penetrate into holes in the support and are secured in these.

A plurality of adjacent anchoring sections can be formed from a single thread- or rod-like structural part, which as a result of corresponding bends and angles forms a large number of U-shaped anchoring sections.

It is particularly advantageous if intersecting anchoring parts form a flat structural element, which is connected to the support at its edges. In this case, the flat structural element forms a structural part that can be independently manipulated and is connected to the support as such.

For connection, the support carries on its side faces a peripheral band, which clamps the edges of the flat structural part against the side faces.

It is advantageous in this case if the side faces have a peripheral groove in the region of the band to receive the edges of the flat structural part.

The anchoring sections can either be embedded into the material of the core from the outset and this composite material is then connected to the support as a unit, or it is provided that the anchoring sections are connected to the support and that the core material is then placed onto the anchoring sections and then envelops the anchoring sections as it swells, i.e. upon absorption of liquid.

In preferred embodiments, it can be advantageous if the anchoring sections are pliable, i.e. configured as bendable threads or wires.

The anchoring sections can be made of metal, preferably of titanium or a titanium alloy.

It can additionally be provided that the anchoring sections are coated with a swellable material, so that an intimate bond occurs between the anchoring sections and the surrounding core material when these anchoring sections are surrounded. In principle, it would also be possible to fabricate the anchoring sections completely from a swellable material, so that the anchoring sections and the surrounding core material are joined together particularly well during swelling of the material.

The anchoring sections can also be made of a hydrophobic polymer, e.g. a drawn polyethylene, which in a particularly preferred form is encased by a hydrophilic polymer that is swellable but not soluble in water.

In another embodiment it is provided that the anchoring sections are made of a hydrogel or a xerogel.

The following description of preferred embodiments of the invention serves as more detailed explanation in association with the drawing:

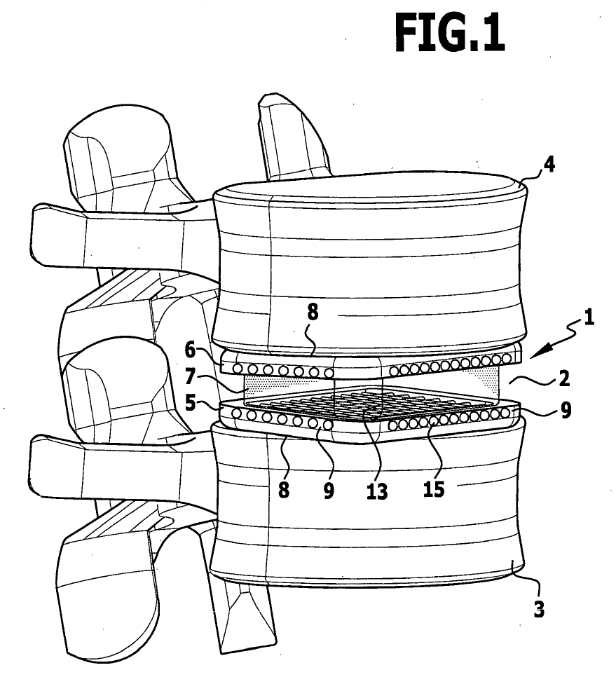

FIG. 1 is a perspective view of two vertebrae with an intervertebral implant inserted between them;

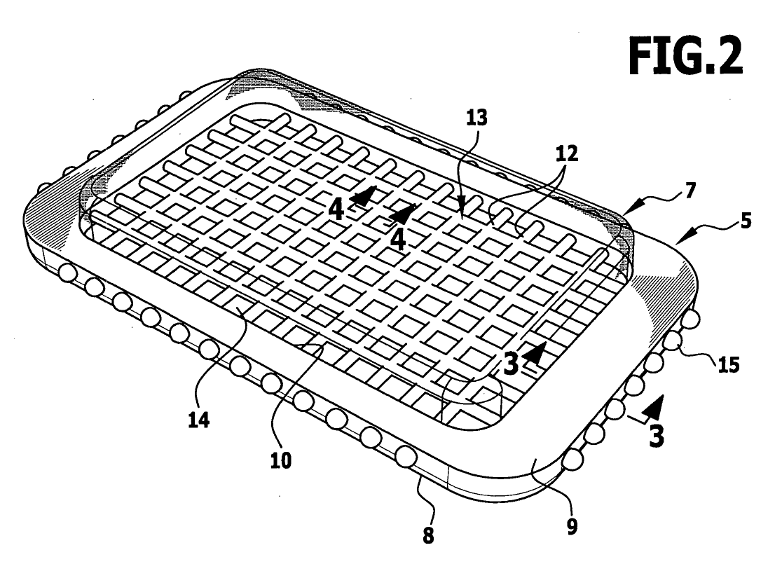

FIG. 2 shows an end plate of the intervertebral implant of FIG. 1 with a core made of a swellable material placed thereon;

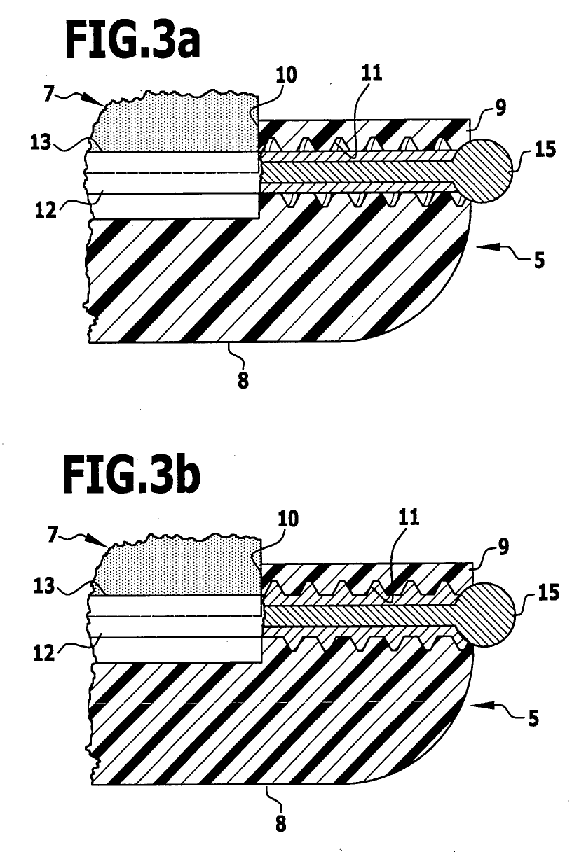

FIG. 3a is a sectional view taken along line 3-3 in FIG. 2 before swelling of a swellable casing of an anchoring section;

FIG. 3b is a view similar to FIG. 3a after swelling of the swellable casing of the anchoring section;

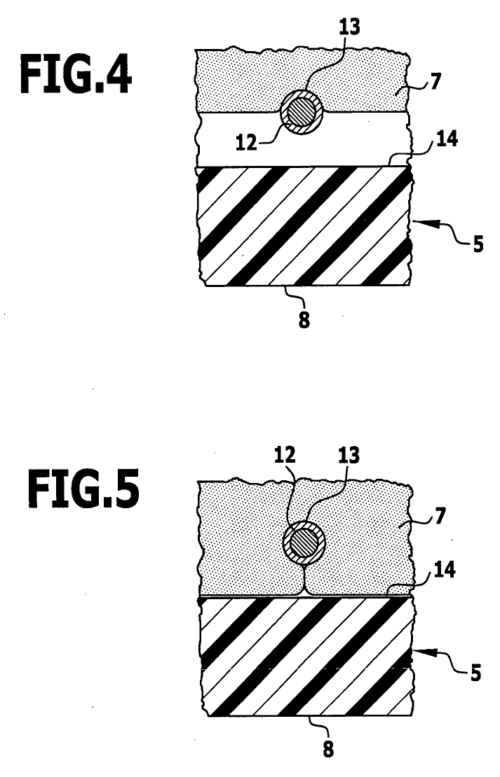

FIG. 4 is a sectional view taken along line 4-4 in FIG. 2 before swelling of the swellable material;

FIG. 5 is a view similar to FIG. 4 after swelling of the swellable material;

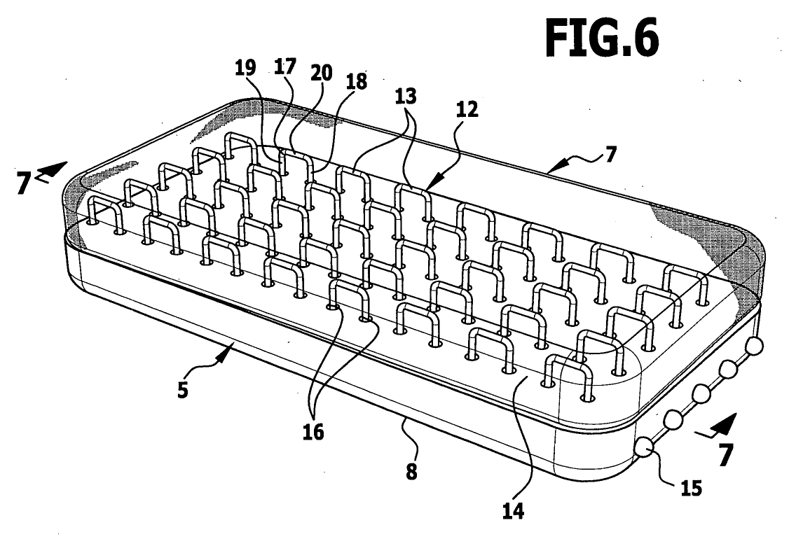

FIG. 6 is a view similar to FIG. 2 with an exemplary embodiment having U-shaped anchoring sections;

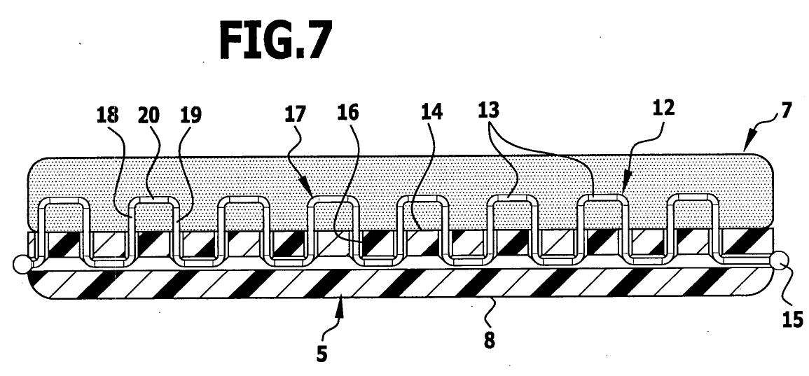

FIG. 7 is a sectional view taken along line 7-7 in FIG. 6;

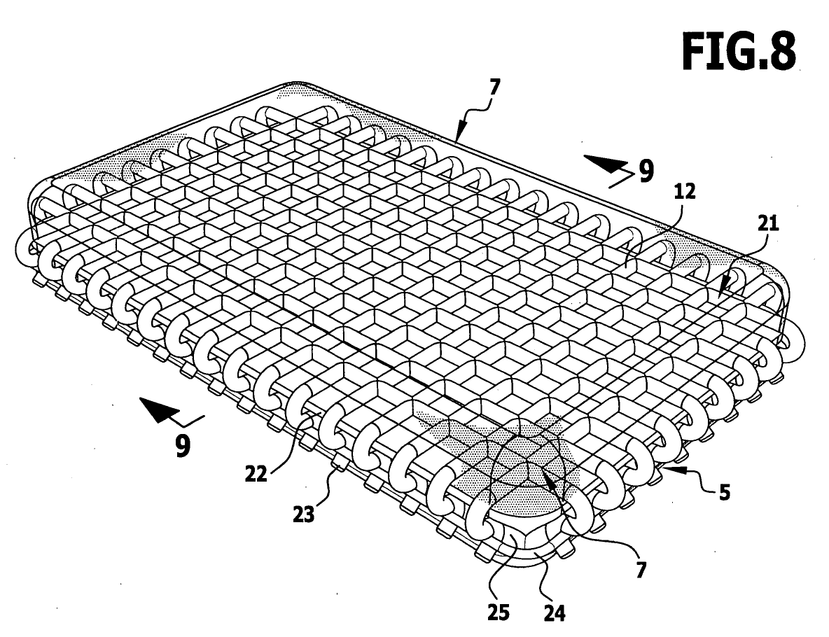

FIG. 8 is a view similar to FIG. 2 with a net-like structural part consisting of anchoring sections and

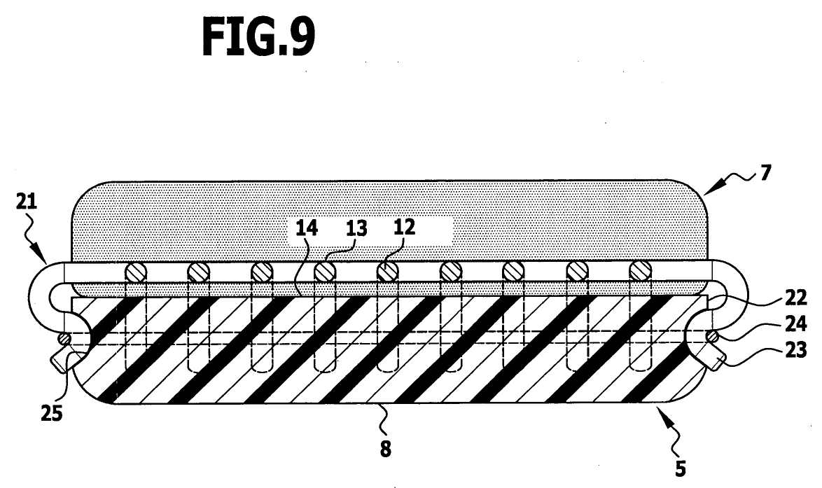

FIG. 9 is a sectional view taken along line 9-9.

In FIG. 1 an intervertebral implant 1 is arranged in the intervertebral space 2 between two adjacent vertebrae 3, 4. It comprises a lower end plate 5 and an upper end plate 6 and a core 7 arranged between these two end plates 5 and 6. The two end plates 5, 6 are configured mirror-inverted to one another, and therefore only the lower end plate 5 will be described in more detail below.

This comprises a plane contact face 8 on its underside and an upwardly projecting frame-like edge 9 on its upper side that is fully closed and surrounds an inside space 10 that is closed to the bottom by the end plate 5 and is open to the top.

The end plate 5 with the upwardly projecting edge 9 is made of a bend-resistant and dimensionally stable biocompatible material, e.g. titanium or a titanium alloy, or, however, also of a thermoplastic, e.g. polyether ether ketone. In the illustrated embodiment, the end plate 5 has a substantially rectangular shape with rounded corners and is adapted in size to the cross-sectional surface of the vertebrae 3, 4, so that the entire intervertebral space is substantially covered by the end plates.

In the embodiment shown in FIGS. 1 to 3, a large number of holes are arranged in the upwardly projecting edge 9 that extend transversely to the longitudinal direction of the edge 9 and are configured as continuous internally threaded holes in the illustrated embodiment. The ends of thread- or wire-shaped anchoring sections 12 penetrate into such respectively opposing holes 11 of the end plate and in this way extend transversely through the entire inside space 10. The anchoring sections 12 of respectively opposing parts of the edge 9 extend parallel to one another, and the anchoring sections 12 of the sections of the edge 9 extending perpendicular to one another accordingly extend perpendicular to one another, i.e. they intersect one another and therefore form a net-like anchoring surface 13. This extends parallel to the end plate 5 at a spacing from its upper side 14 and in the inside space 10. In this case, the entire inside space is uniformly covered by anchoring sections 12, as may be seen from the representation in FIG. 2.

The anchoring sections 12 are firmly connected to the edge 9 at their ends penetrating into the holes 11. As may be seen from FIGS. 3a and 3b, the anchoring sections 12 can have a coating of swellable material, which increases in volume upon absorption of water. It can only be the casing in this case. However, it can also be provided that the anchoring section is composed completely of such a material. The ends of the anchoring sections 12 configured in this way are arranged in an internally threaded hole in the region of the edge 9. Before the swellable casing swells, the swellable material does not penetrate into the thread turns of the internally threaded hole because of its small volume (FIG. 3a), i.e. the outside diameter of the anchoring sections corresponds to the smallest inside diameter of the internally threaded hole, so that the anchoring sections can be inserted into the internally threaded hole.

After fluid absorption, the volume of the swellable casing material or of the swellable material of the anchoring sections increases in such a manner that this material enters the thread turns and more or less completely fills these, as is shown in FIG. 3b. A positive-locking anchorage of the anchoring sections in edge 9 results from this.

However, the anchoring sections 12 can also be secured in the edge 9 exclusively or additionally by enlargements 15, which abut against the outside of the edge 9 and clamp the anchoring sections 12 between the opposing parts of the frame 9.

The anchoring sections 12 are made of a material in the form of a thread, in particular a pliable thread, or in the form of a wire or of a very thin elongated rod, so that a gap remains between the upper side 14 of the end plate 5 and the underside of the anchoring sections 12 (FIG. 4).

The anchoring sections can preferably be made of metal, in particular of titanium or a titanium alloy. However, other materials can also be used, e.g. hydrophobic polymers such as drawn polyethylene, preferably high molecular weight polyethylene, in particular ultrahigh molecular weight polyethylene (UHMWPE) or an incompletely expanded hydrogel, or the anchoring sections can be made of a hydrogel or a xerogel, i.e. a pure water-free polymer. The anchoring sections 12 can be configured as a multifilament or monofilament structure, and it is possible that the anchoring sections 12 are additionally coated, e.g. with a swellable xerogel, which forms a hydrogel upon contact with the body fluid present.

The plate-shaped core, which is adapted in its cross-section to the cross-section of the inside space 10, is placed on the anchoring surface 13 configured by the anchoring sections 12, so that its underside lies on the anchoring sections 12. The identically configured upper end plate 6 can be laid on the upper side of the intervertebral implant 1 and is then supported on the upper side of the core 7 at its anchoring sections 12.

The core is made of a swellable hydrogel, i.e. a material that can increase in volume on fluid absorption.

In principle, all non-degradable, hydrophilic polymers are conceivable as hydrogels. Examples are polyacrylic acid and its derivatives such as polymethacrylic acid, polyacrylamide, polyacrylonitrile, polyacrylate, polyhydroxy ethyl methacrylates, and additionally polyvinyl pyrrolidone (PVP), polyurethanes, high molecular weight polyvinyl alcohol.

Polymer blends (copolymers that are interconnected by covalent bonds) of the above-mentioned polymers or interpenetrating networks (IPNs) of the above-mentioned polymers are also conceivable. IPNs consist of at least two different polymers, the polymer chains of which are entangled and are interconnected by physical interactions (van der Waals, electrostatic, H bridge-ring compounds and/or ionic forces).

Further polymer mixtures that can be used are copolymers as well as IPNs of polyacrylates (polyacrylic acid and its derivatives such as polymethacrylic acid, polyacrylamide, polyacrylonitrile, polyacrylate) with polycaprolactone.

The swellable material of the core 7 increases in volume upon fluid absorption, i.e. after implantation into the intervertebral space 2, and in so doing the material of the core 7 surrounds the anchoring sections 12 of the anchoring surface 13 on all sides, i.e. the entire inside space 10 of the two end plates 5, 6 is completely filled, as is shown in FIG. 5. As a result of this, the anchoring sections 12 are embedded into the material of the core 7 and thus the core interlocks with the adjacent end plates.

This interlocking can be further improved if not only a positive-locking connection is configured between the material of the anchoring sections and the material of the core, but in addition flexible electrostatic, ionic or van der Waals interactions are used to anchor the core to the anchoring sections. In this case, the properties of the material of the core 7, in particular the swelling capability, can be varied within certain limits by different parameters of the swellable material, e.g. by using a different molecular weight, by a different cross-link density or by a different production process.

In particular, a particularly good cross-linkage results with anchoring sections 12 that are themselves coated with a polymer. This can be the same polymer material as the material of the core, but different polymer materials that intimately cross-link with one another can also be used.

In the embodiment of FIGS. 1 to 5 the anchoring sections 12 are present in the form of a net-like anchoring surface 13.

Other geometric arrangements of the anchoring sections 12 on the end plates 5, 6 are also possible, it is merely important that the swellable material has the possibility of enclosing the anchoring sections on all sides.

In the embodiment of FIGS. 6 and 7, a wire-like structural element embedded into the end plate 5 is directed upwards and downwards again in the form of a loop through adjacent holes 16 in the upper side 14 of the end plate 5, so that bridges or loops 17 with a U-shaped cross-section, which have parallel legs 18, 19 exiting perpendicularly upwards from the holes 16 and bars 20 connecting these and extending parallel to the upper side 14 and at a spacing from this, are configured above the upper side 14 of the end plate 5. A large number of such rows of loops 17 are arranged next to one another on the upper side 14 of the end plate 5 and thus cover the entire upper side 14. In this case, the bars 20 extending parallel to the upper side 14 form an anchoring surface 13, onto which the core 7 is placed in a similar manner to that in the embodiment of FIGS. 1 to 5. Upon swelling the material of the core then surrounds the bars 20 on all sides and also the legs 18, 19 projecting out of the upper side 14, so that a firm and secure connection is assured between the core 7 and the end plate 5.

The looped structural elements can be configured as wire elements, i.e. can be made of a metal material, in particular of titanium or a titanium alloy. However, polymer threads that are preferably rigid to bending in this case, e.g. polyethylene threads, can also be used.

A further possible configuration is shown in the embodiment of FIGS. 8 and 9. There, anchoring sections 12 extending parallel and intersecting one another are combined to form an anchoring surface 13, which is configured as an independently manipulated structural part. For this purpose, the anchoring sections can be woven together, for example, or be interconnected in a suitable manner at the intersection points. In the edge region this independently manipulated net-like structural part 21 is configured with downwardly bent edges, i.e. the ends of the anchoring sections 12 are bent downwards, so that they come into abutment against the side faces 20 of the end plate 5 from the outside when the structural part 21 is placed on the end plate 5 to cover it (FIG. 8). The ends 23 of the anchoring sections 12 are then pressed firmly against the side faces 22 by a peripheral band 24, so that the structural part 21 is permanently secured to the end plate 5 thereby. The fixture is assisted by a peripheral groove 25 being machined into the side face 22 that extends around the entire end plate 5 along the side faces 22, the ends 23 being pressed into said groove by the band 24 (FIG. 9).

In a similar manner to that in the above-described practical examples, the core 7 is placed on the structural part 21, and then surrounds the anchoring sections 12 on all sides upon swelling and a firm connection with the end plate 5 results.

In principle, it would also be possible to connect the core 7 to the structural part 21 already before mounting the structural part 21 on the end plate 5, i.e. to embed the structural part 21 in the core 7 already during production. A core with a structural part 21 embedded in this manner can then be placed onto the end plate 5 and be secured to the end plate 5 by the band 24. Thus, a connection is already achieved before the swellable material swells in the body.

What is claimed is:

1. Intervertebral implant with at least one contact element having a vertebral body contact face and with a swellable core, which is connected to the contact element on the side remote from the vertebral body contact face, wherein the contact element comprises a support, on one side of which the vertebral body contact face is arranged and which on its other side carries projections surrounded by the material of the core, wherein the projections have anchoring sections, which are elongated, thread-like or rod-like, and which extend at a spacing from the support and are freely accessible from all sides.

2. Implant according to claim 1, wherein the anchoring sections extend substantially parallel to the vertebral body contact face and at a spacing from the side of the support remote from the vertebral body contact face.

3. Implant according to claim 1, wherein substantially the entire surface of the support has anchoring sections.

4. Implant according to claim 1, wherein a plurality of anchoring sections extend parallel to one another.

5. Implant according to claim 1, wherein various anchoring sections intersect.

6. Implant according to claim 5, wherein intersecting anchoring sections form a net-like structure.

7. Implant according to claim 1, wherein the support has frame-like side parts; on which the anchoring sections are held.

8. Implant according to claim 7, wherein the anchoring parts penetrate into holes of the frame-like side parts and are secured in these.

9. Implant according to claim 8, wherein the anchoring parts are screwed into the holes.

10. Implant according to claim 8, wherein the anchoring parts are secured in the holes by means of enlargements or knots on the outside of the side parts.

11. Implant according to claim 8, wherein the anchoring parts are made of swellable material and surround spigots and sockets in the holes upon swelling.

12. Implant according to claim 1, wherein the anchoring sections are U-shaped with two legs, which extend transversely to the support and secure the anchoring sections on the support and with a web connecting these legs.

13. Implant according to claim 12, wherein the legs and the web extend substantially rectilinearly.

14. Implant according to claim 12, wherein the legs penetrate into holes in the support and are secured in these.

15. Implant according to claim 12, wherein a plurality of adjacent anchoring sections are formed from a single thread- or rod-like structural part.

16. Implant according to claim 5, wherein intersecting anchoring parts form a flat structural element, which is connected to the support at its edges.

17. Implant according to claim 16, wherein on its side faces the support carries a peripheral band, which clamps the edges of the flat structural part against the side faces.

18. Implant according to claim 17, wherein in the region of the band the side faces have a peripheral groove to receive the edges of the flat structural part.

19. Implant according to claim 1, wherein the anchoring sections are pliable.

20. Implant according to claim 1, wherein the anchoring sections are made of metal.

21. Implant according to claim 20, wherein the anchoring sections are made of titanium or a titanium alloy.

22. Implant according to claim 20, wherein the anchoring sections are coated with a swellable material.

23. Implant according to claim 1, wherein the anchoring sections are made of a hydrophobic polymer.

24. Implant according to claim 23, wherein the anchoring sections are made of polyethylene.

25. Implant according to claim 1, wherein the anchoring sections are made of a hydrogel or a xerogel.
